(19) United States
(12) Patent Application Publication
(10) Pub. No.: US 2026/0325809 A1
(43) Pub. Date: Oct. 22, 2026

(54) Microneedle Patch with Force-Feedback Indicator

(71) Applicant: Micron Biomedical, Inc., Atlanta, GA (US)

(72) Inventors: Devin V. McAllister, Marietta, GA (US); Charles M. Quillian, Stone Mountain, GA (US); Rolando H. Galvez-Gallardo, Dallas, GA (US)

(21) Appl. No.: 19/111,250

(22) PCT Filed: Sep. 13, 2023
(86) PCT No.: PCT/US2023/032664

§ 371 Date: Mar. 12, 2025

Related U.S. Application Data
(60) Provisional application No. 63/405,932, filed on Sep. 13, 2022.

(51) Int. Cl. A61M 37/00

(52) U.S. Cl. CPC A61M 37/0015; A61M 2205/581; A61M 2205/582; A61M 2209/06

(57) ABSTRACT

A microneedle patch is provided that includes a microneedle array; a base substrate from which the microneedles extend; and a force feedback indicator (FFI) attached to the base substrate. The FFI includes a base and a button, which has an upper surface and a side surface, wherein the button is configured to translate from a pre-actuated position into an actuated position within the base, wherein the side surface is substantially visible in the pre-actuated position and substantially not visible in the actuated position. The side surface has different color than the upper surface and/or base, which facilitates visual confirmation of the actuation status of the button, which can help indicate whether the microneedle patch is ready for use or has been used. The FFI may be configured to provide tactile, audible, and visual confirmation of sufficient force applied to microneedle patch to effect insertion of the microneedles.

### CROSS-REFERENCE TO RELATED APPLICATIONS

This application claims priority to U.S. Provisional Application No. 63/405,932, filed Sep. 13, 2022, which is incorporated herein by reference.

### BACKGROUND

The present application is generally in the field of microneedle patches for the administration of bioactive agents or other suitable substances into a biological tissue, for example, to administer a vaccine, contraceptive, or other drug into a person's skin.

It would be desirable to provide improved microneedle patches that can simplify and improve the delivery of vaccines and other agents, that store the agent in a dry form, that can be easily and consistently be applied manually, e.g., without requiring a separate applicator device, that can deliver the agent payload into the skin in a short duration, e.g., such that the patch can be removed from the skin within a few minutes of being applied to the skin, that leaves no microneedle sharp following administration of the agent payload, and that include feedback indicators to confirm that sufficient manual force has been applied to the patch to insure complete or proper insertion of the microneedles into the skin, and/or that includes an indicator to show that the microneedle patch has been used.

It would also be desirable to provide new and improved systems for packaging and protecting the microneedles of the microneedle patch before use. In particular, it may be important to provide such systems in a compact design, where the microneedle patch products must be kept cold during transportation and storage before use.

### BRIEF SUMMARY

In one aspect, a microneedle patch is provided that includes: an array of microneedles; a base substrate having a first side from which the microneedles extend and an opposing back side; and a force feedback indicator (FFI) attached to the back side of the base substrate, wherein the FFI comprises a base and a button, which has an upper surface and a side surface, wherein the button is configured to translate from a pre-actuated position into an actuated position within the base, wherein the side surface is substantially visible in the pre-actuated position and substantially not visible in the actuated position. The FFI preferably is configured to further provide tactile and/or audible confirmation of sufficient force applied to microneedle patch to effect insertion of the microneedles. In a particular embodiment, the side surface has different color than the color of the upper surface and/or the color of the base, which facilitates visual confirmation of the actuation status of the button, which can help indicate whether the microneedle patch is ready for use or has been used.

In another aspect, a microneedle patch is provided that includes: an array of dissolvable microneedles; a base substrate having a first side from which the microneedles extend and an opposing back side; and a wear time indicator (WTI) which is attached to the back side of the base substrate and which is configured to provide a visual indication that the microneedle patch has been worn on a user's skin for a period sufficient to effect dissolution of the microneedles following their insertion into the user's skin. (The period is a wear time.) In a particular embodiment, the WTI includes a dye blister and a wick assembly, wherein the dye blister has a burstable dye reservoir configured to release a dye into the wick assembly upon application of a force to the microneedle patch to effect insertion of the microneedles. The position of the dye within the wick assembly corresponds to the wear time.

In a further aspect, a microneedle patch is provided that includes: an array of microneedles; a base substrate having a first side from which the microneedles extend and an opposing back side; and a force feedback indicator (FFI) comprises a base and a button, wherein the base substrate and array of microneedles are attached only to the button of the FFI, wherein the button is configured to translate from a pre-actuated position into an actuated position within the base, and wherein the base of the FFI is dimensioned such that in the pre-actuated position, the array of microneedles is in a position recessed in an opening in a lower surface of the base of the FFI. In a particular embodiment, at least an upper portion of the button in the pre-actuated position is elevated above the base and in the actuated position is flush with or recessed into the base. The button may have an upper surface and a side surface, wherein the side surface is substantially visible in the pre-actuated position and substantially not visible in the actuated position. In some embodiments, the button of the FFI includes a latch, and the housing includes (i) a first latch receptacle, which is configured to receive the latch and releasably hold the button in the pre-actuated position, and (ii) a second latch receptacle, which is configured to receive the latch and unreleasably hold the button in the actuated position. In a particular embodiment, the microneedle patch is configured such that a first minimum force on the button is effective to dislodge the latch from the first latch receptacle and displace the button toward the base and begin insertion of the microneedles into a tissue surface; and such that a second minimum force, which may be greater than the first minimum force, on the button is effective to move the latch into the second latch receptacle and trigger a tactile signal and/or an audible signal that sufficient force has been applied to the microneedle patch to effect full insertion of the microneedles into the tissue surface.

In still another aspect, microneedle patch packaging units and systems are provided. In one embodiment, a microneedle patch packaging system is provided that includes (i) a microneedle patches which has an adhesive surface; and (ii) a foil or other pouch material adhered to the adhesive surface, wherein the foil or other pouch material is folded over and sealed to form a sealed pouch enclosing the at least one of the microneedle patches. In another embodiment, a microneedle patch packaging unit is provided that includes (i) a microneedle patch which has a handling tab; (ii) a packaging tray having a cavity in which the microneedle patch is disposed; and (iii) a foil or other film attached to the tray to seal the cavity, wherein the packaging system is configured such that upon removal of the foil or other film from the tray, the handling tab is positioned toward the opening of the cavity to facilitate ease of grasping the handling tab to remove the microneedle patch from the packaging tray. In some embodiments, this favorable handling tab position may be achieved by a design in which the handling tab is folded over a top of the of the microneedle patch within the sealed tray and partially unfolds itself upon removal of the foil or other film from over the cavity. In another embodiment, a packaging system is provided that includes comprising a plurality of these microneedle patch packaging units, wherein an edge of the packaging tray of each packaging unit is releasably attached at an edge of at least one other packaging tray of another packaging unit, for example, wherein the releasably attached edges defined by a line of perforations in a shared sheet material.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components are not necessarily drawn to scale.

FIGS. 1A and 1B are perspective and exploded views, respectively, of a microneedle patch with a FFI, according to one or more embodiments of the present disclosure.

FIG. 2A is a side view of a microneedle patch with a FFI, according to one or more embodiments of the present disclosure.

FIG. 2B is an upper perspective view of the microneedle patch shown in FIG. 2A, according to one or more embodiments of the present disclosure.

FIG. 2C is a bottom view (microneedle array side) of the microneedle patch shown in FIG. 2A, according to one or more embodiments of the present disclosure.

FIG. 2D is a top view (button side) of the microneedle patch shown in FIG. 2A, according to one or more embodiments of the present disclosure.

FIG. 3A is a perspective view of a storage tray for a microneedle patch, according to one or more embodiments of the present disclosure.

FIG. 3B is a bottom, perspective view of the storage tray of FIG. 3A with a microneedle patch stored therein, according to one or more embodiments of the present disclosure.

FIG. 3C is a top, perspective view of the storage tray of FIG. 3A with the microneedle patch stored therein, according to one or more embodiments of the present disclosure.

FIG. 4A is a top, perspective view of a microneedle patch with the upper portion of the button of the FFI in the pre-actuated position, elevated above the base of the FFI, according to one or more embodiments of the present disclosure. The downwardly directed arrow illustrates a force to be applied to the button.

FIG. 4B is a top, perspective view of a microneedle patch shown in FIG. 4A, but with the upper portion of the button of the FFI in the actuated position slightly recessed into, the base of the FFI, according to one or more embodiments of the present disclosure.

FIG. 5A is a side, cross-sectional view of a microneedle patch with the button of the FFI in the pre-actuated position, elevated above the base of the FFI, and with the microneedle array recessed within the base, according to one or more embodiments of the present disclosure.

FIG. 5B is a side, cross-sectional view of the microneedle patch of FIG. 5A, but with the button of the FFI in the actuated position recessed into the base of the FFI, and with the microneedle array extending out from the base, according to one or more embodiments of the present disclosure.

FIGS. 6A-6C are microphotographs of dissolving microneedles, according to one or more embodiments of the present disclosure.

FIGS. 7A-7C depict a process of using a microneedle patch in which its microneedles are inserted into a biological tissue and dissolve and separate from the patch backing, according to one or more embodiments of the present disclosure.

FIG. 8 depicts a step in a molding process in which droplets are placed onto a mold for a segmented microneedle array, according to one or more embodiments of the present disclosure.

FIG. 9 is a bottom, perspective view of a microneedle patch with a segmented microneedle array, according to one or more embodiments of the present disclosure.

FIG. 10A is a perspective view of a packaging unit that includes microneedle patch stored in a rectangular storage container (tray) and enclosed by a removable cover material, with the storage container and cover material being transparent and shown in broken lines, according to one or more embodiments of the present disclosure.

FIG. 10B is perspective view of a packaging system comprising ten of the packaging units shown in FIG. 10A, nine units of which are attached to at least one other unit along interfacing side edge, according to one or more embodiments of the present disclosure. The other unit is shown detached, having been separated from the others along a line of perforations.

FIG. 10C is a perspective view of a further packaging system comprising a boxed stack of five of the packaging systems shown in FIG. 10B, with the box being transparent and shown in broken lines, according to one or more embodiments of the present disclosure.

FIG. 11A is a perspective view of another packaging system comprising a plurality of microneedle packaging units, each comprising a trapezoidal shaped storage tray, and wherein each unit is shown attached to at least one other unit along interfacing side edge, according to one or more embodiments of the present disclosure.

FIG. 11B is a perspective view of an opened one of the microneedle packaging units shown in FIG. 11A, with the microneedle patch disposed with the cavity of the tray (with the cover material removed), according to one or more embodiments of the present disclosure.

FIG. 12 is an exploded view of a wear time indicator for a microneedle patch, according to one or more embodiments of the present disclosure.

FIGS. 13A-13D illustrates a time-lapse of the wear time indicator of FIG. 12 in use, according to one or more embodiments of the present disclosure.

### DETAILED DESCRIPTION

Improved microneedle patches and systems have been developed to provide enhanced usability, convenience, handling/storage capability, and/or functionality.

The microneedle patches include an array of microneedles extending from a base substrate, which is connected to other components facilitating handling and insertion of the microneedles. Those components typically include a tape layer comprising an adhesive face and a handling tab, and may further include a force feedback indicator (FFI) or a wear time indicator (WTI). In particular embodiments, the FFI and WTI are not electronic. Instead, they are operate mechanically, and typically are made from relatively inexpensive polymeric parts that can be produced in high-volume manufacturing processes.

In some preferred embodiments, the microneedles comprise a substance of interest and a water-soluble matrix material in which the substance of interest is dispersed.

In some embodiments, a force feedback indicator (FFI) attached to the back side of the base substrate, wherein the FFI comprises a base and a button, which has an upper surface and a side surface, wherein the button is configured to translate from a pre-actuated position into an actuated position within the base, wherein the side surface is substantially visible in the pre-actuated position and substantially not visible in the actuated position. The FFI preferably is configured to further provide tactile and/or audible confirmation of sufficient force applied to microneedle patch to effect insertion of the microneedles.

In some embodiments, the microneedle patch includes an array of microneedles; a base substrate having a first side from end the microneedles extend and an opposing back side; a tape layer comprising an adhesive face and a handling tab; and a force feedback indicator (FFI), which is secured to the tape layer. The FFI may be configured to provide an audible, tactile, and/or visual signal when a force applied to the patch by a user, in the course of applying the patch to a biological tissue to insert the solid microneedles into the biological tissue, meets or exceeds a predetermined threshold. The tape layer may be a double-sided adhesive tape, a plastic film with adhesive disposed on either or both sides, or a double sided tape.

In some embodiments, the tape layer comprises an aperture through which a central portion of the base extends, and the microneedle array is mounted on this central portion to elevate the microneedles off of the surrounding tape layer and away from its adhesive face, which facilitates insertion of the microneedles when the adhesive face is pressed against and adhered to a skin surface. The elevated microneedles may also provide the force necessary to maintain the microneedle array within the skin (i.e., provides a hold-down force on the microneedles) for the duration of the patch wear time.

Some examples of suitable microneedle arrays and their methods of manufacture that may be used with the present microneedle patches and packaging system are described in U.S. Pat. Nos. 10,265,511; 10,828,478; 10,828,478; 10,940,301; and US20200238065A1, which are incorporated herein by reference.

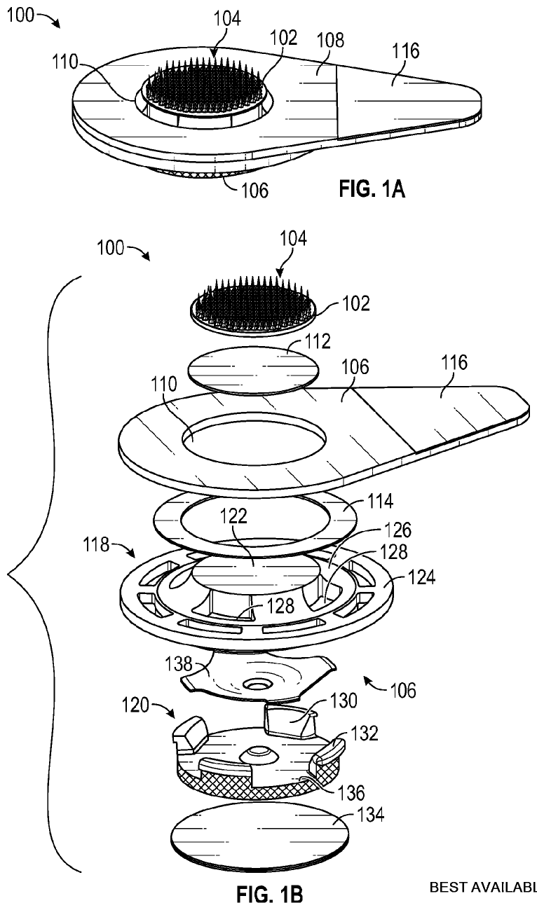

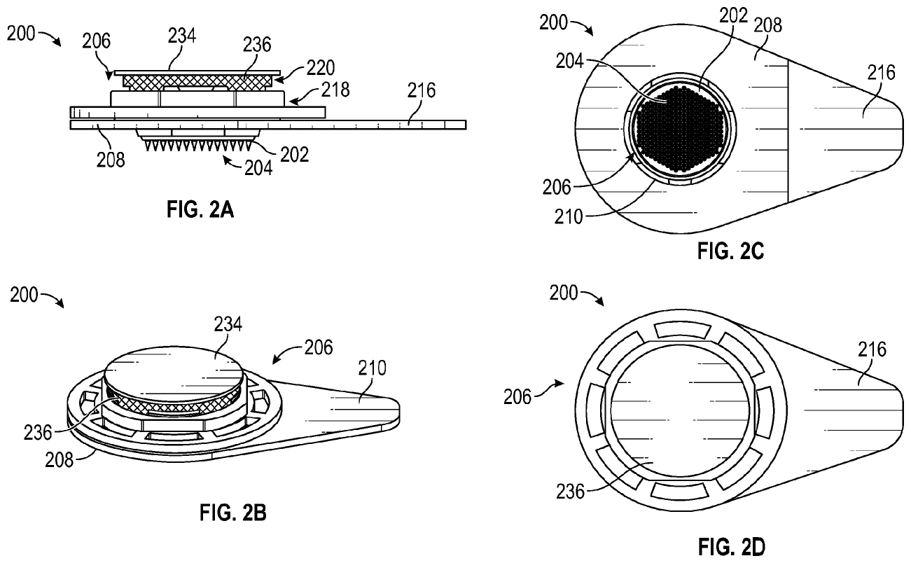

The microneedle patches may include an array comprising any suitable number of microneedles, e.g., from 10 to 10,000 microneedles such as from 50 to 1000 microneedles. The periphery of the array may have an outer shape that is circular (e.g., as shown in FIG. 1) or hexagonal (e.g., as shown in FIGS. 2A-2D) both of which may extend from a base that is circular or another shape.

In a preferred embodiment, the microneedles are solid microneedles that include a substance of interest, such as an active pharmaceutical ingredient (API), which becomes solubilized in vivo following insertion of the microneedle into a biological tissue, e.g., into the skin of a patient. For example, the substance of interest may be mixed in a water soluble matrix forming the solid microneedle extending from the base substrate, or the substance of interest may be in the form of a coating on a microneedle sub-structure extending from the base substrate. In either case, the substance of interest may be provided in a formulation referred to herein as being “dissolvable.” In embodiments in which the substance of interest and a matrix material in which the substance of interest is dispersed form the structure of the microneedle, the matrix material also preferably is dissolvable in vivo, such that the entire portion of the microneedle inserted into the biological tissue dissolves in vivo (e.g., about 90% to 95% of the total length of the microneedle). In embodiments in which the substance of interest is part of a coating on a microneedle sub-structure, the sub-structure may also be dissolvable in vivo.

The microneedles may have a height from about 100 μm to about 2000 μm, from about 100 μm to about 1500 μm, from about 100 μm to about 1000 μm, or from about 500 μm to about 1000 μm. The microneedles may be arranged on a base substrate in any suitable density.

### Microneedle Patches with Force Feedback Indicators (FFI)

An exemplary microneedle patch with a FFI and a plurality of solid microneedles is illustrated in FIG. 1. The patch 100 includes a base substrate 102 with a plurality of microneedles 104. The plurality of microneedles 104 is attached to a FFI 106. The microneedles 104 and FFI 106 may be attached to a backing layer 108 via an opening 110 therein. That is, the backing layer 108 may include an opening 110 sized and shaped to receive the plurality of microneedles 104 and FFI 106 within the opening 110. In some embodiments, the base substrate 102 holding the plurality of microneedles 102 is attached to the FFI 106 with a first adhesive layer 112, and the FFI 106 is attached to the backing layer 108 by a second adhesive layer 114. In other embodiments, the base substrate 102 and the backing layer 108 are integrally formed with the FFI 106.

In some embodiments, the backing layer 108 may include a tab portion 116 which extends laterally away from the microneedles 104. Alternatively, the tab portion may be disposed in a separate layer (not shown). Thus, the tab portion may be in the same plane or in a different plane than the backing layer. The “backing layer” and the “handle layer” may be used interchangeably in the present disclosure unless expressly provided otherwise. The tab portion 108 may advantageously enable a patient or user to handle the patch 100 without contacting the “body portion” of the patch defined by the base substrate 102 and the plurality of microneedles 104. For example, the tab portion 116 may be sized and shaped to permit a person to manually hold the tab portion 116 (e.g., between a thumb and finger). Although the tab portion 116 is illustrated in FIG. 1 as extending laterally and asymmetrically from the backing layer 108, other shapes and sizes are also possible.

In some embodiments, an adhesive (not shown) is disposed on the microneedle 104 side of the backing layer 108 to help adhere the patch 100 to the patient's skin during application. The adhesive may also function to adhere the patch to a tray or container covering the plurality or microneedles during shipping and storage, as well as for disposal after its use. In one embodiment, the tab portion 116 is substantially free of the adhesive layer, enabling a person handling and applying the patch to do so without contacting the adhesive layer. In some embodiments, the adhesive layer may be disposed over substantially all of a side of the backing layer 108, including the tab portion 116. A cover portion (not shown) may be disposed over the adhesive layer over the tab portion 116 so that a person holding the patch 100 by the tab portion does not contact much of the adhesive layer.

The FFI 106 includes a base 118 and a button 120 configured to translate within the base 118. The base 118 may include a center portion 122, an outer portion 124, and an intermediate portion 126 positioned between and connecting the center portion 122 and the outer portion 124. The base substrate 102 carrying the microneedles 104 is attached to the center portion 122 of the base 118 via the first adhesive layer 112. The outer portion 124 of the base 118 may be attached to the back side (i.e., the side opposite the microneedles) of the backing layer 108 via the second adhesive layer 114. That is, the second adhesive layer 114 may have a ring-like shape so that the second adhesive 114 may be placed around the center portion 122 and the intermediate portion 126 and onto the outer portion 124.

The button 120 may be slidably attached to the base 118 through one or more slots 128 disposed around the circumference of the intermediate portion 126 of the base 118. That is, relative to FIG. 1, the button 120 may have one or more upwardly extending protrusions 130 configured to be received within the one or more slots 128 of the base 118. Each of the one or more protrusions 130 may also include a lip 132 to secure the protrusions 130 within the slots 128, which may prevent unintended removal of the button 120 from the base 118.

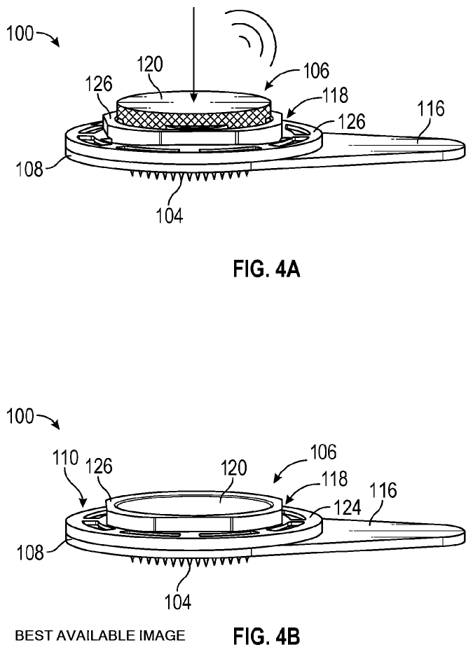

The button 120 may also include an upper surface 134 and a side surface 136. The button 120 may translate from a pre-actuated position to an actuated position, where the side surface 136 is visible in the pre-actuated position and substantially not visible in the actuated position. For example, as shown in FIGS. 4A-4C, when the patch 100 is assembled the button 120 may initially sit proud above the backing layer 108 and the base 118 of the feedback indicator. Upon the application of a downward force to the top of the button 120 (i.e., a patient or user depressing the button 120), the button 120 may move downward into the central portion 122 of the base 118. When the button 120 is fully translated from the pre-actuated position to the actuated position, such that the button 120 is fully disposed within the base 118 and no longer visible. In some embodiments, the side surface 136 may be formed of a material having a different color from the rest of the feedback indicator 106, which may help the user to better identify when the button 120 has fully translated from the pre-actuated position to the actuated position, indicating that the plurality of microneedles 104 have been at least partially inserted into the tissue.

The FFI 106 also includes a secondary mechanism for providing a user with feedback to assist with the proper and effective use of the microneedle patch. For example, in some instances, the translating the button 120 into the actuated position may not be sufficient to fully insert the microneedles 104 into the tissue. That is, actuating the button 120 from the pre-actuated position to the actuated position may only be sufficient to penetrate the tissue and partially insert the microneedles 104, requiring additional force to fully insert the microneedles 104 into the skin. In other instances, it may be beneficial to utilize a secondary feedback mechanism so that the patient or user is assured that the microneedles have been fully inserted. The secondary feedback may be provided in a variety of forms or combinations, including tactile (e.g., detectable sensation felt by the person administering the patch or the patient), audible (e.g., the presence, absence, or change of sound).

The feedback may be provided to a variety of “users”, including but not limited to the person (e.g., a patient) to whom the microneedle patch is applied, as well as any other person (health care worker, caregiver, parent, guardian) applying the microneedle patch to the person.

In preferred embodiments, the FFI indicates to the user the amount of force and/or pressure applied to the patch during its administration. For example, in one embodiment, the indicator is configured to provide a signal when a force applied to the patch by a user (in the course of applying the patch to a patient's skin to insert the microneedles into the patient's skin) meets or exceeds a predetermined threshold. For instance, the predetermined threshold may be the minimum force or some amount greater than the minimum force that is required for a particular microneedle patch to be effectively applied to a patient's skin. That is, it is the force needed to cause the microneedles to be properly, e.g., substantially, inserted into a patient's skin.

The FFI can signal to the user in a variety of different ways that the predetermined threshold has been met or exceeded. In one embodiment, the FFI may change from its initial configuration to its signaling configuration upon receiving a force which meets or exceeds the predetermined threshold.

In some embodiments, the FFI 106 also include a snap dome 138 disposed within the button 120, which may be designed to collapse (deform) upon application of a sufficient force, which meets or exceeds the predetermined threshold. The collapse may emit a snapping sound and/or can be felt by the user's finger used to apply the patch. In this way, the snap dome provides tactile, visual, and audible signals to the user that the threshold force is met or exceeded and that the patch has been properly applied to the patient's skin. The snap dome may be a bi-stable snap dome. The FFI preferably is configured to undergo irreversible displacement by integrating the snap dome with other components, such as the button and base which become latched together as described herein.

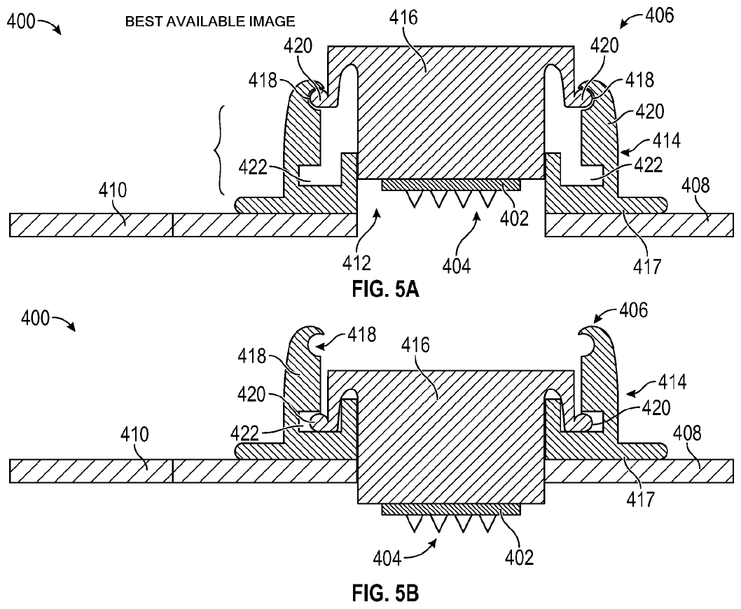

Another microneedle patch with an integrated FFI is shown in FIGS. 5A-5B. The microneedle patch 400 includes a base substrate 402 with an array of microneedles 404. The base substrate 402 is attached to only to the button 416 of the FFI 406 at a bottom surface of the button 416. The base substrate 402 may be attached with an adhesive layer (not show) or the base substrate 402 may be integrally formed with the button 416.

The microneedle patch 400 also included a backing layer 408 attached to the base 114 of the FFI 406 at a lower surface 417 of the base and an upper surface of the of the backing layer. For example, the base of the FFI may be attached via an adhesive disposed on the top side of the backing layer 408. In some embodiments, the backing layer 408 comprises an adhesive tape (e.g., a film or other thin structure comprising a polymeric support/base layer and an adhesive layer (such as a pressure sensitive adhesive known in the art) disposed thereon. In this way, the FFI 406 may be directly attached to the backing layer 408.

The backing layer 406 includes a tab portion 410 which extends laterally away from one side of the base 414 to help a user to handle the patch 400 without contacting the base or the microneedles 404. However, the microneedles 404 advantageously are disposed within a recess 412 defined with the base 414 (and an opening in the backing layer 408) to further protect the microneedles from unwanted contact with anyone or anything until an intended insertion of the microneedles into skin or another tissue surface.

In use, the array of microneedles 404 translates with the button 416, through/from the recess 412 for insertion. To apply the microneedle array 404, the button 416 is depressed downwardly and displaced from a pre-actuated position where the microneedles 404 are disposed within the recess 412, to an actuated position where the microneedles 404 protrude from the recess 412.

In the pre-actuated position, the button 416 is held in place within the base 414 with a latch 420 which extends laterally from the button 416 and is received in a first latch receptacle 418 in the housing. The latch 420 releasably holds the button 416 in the pre-actuated position as shown in FIG. 5A. The housing (base) also includes a second latch receptacle 422, which is configured to receive the latch 420 and hold, preferably unreleasably, the button in the actuated position. The latch and receptacles may be designed such that a first minimum force on the button is effective to dislodge the latch from the first latch receptacle and displace the button toward the base and begin insertion of the microneedles into a tissue surface, and a second minimum force on the button is effective to move the latch into the second latch receptacle and trigger a tactile signal and/or an audible signal that sufficient force has been applied to the microneedle patch to effect full insertion of the microneedles into the tissue surface. In some preferred embodiments, the first minimum force is less than the second minimum force.

In some other embodiments, the first minimum force on the button is effective both (i) to dislodge the latch from the first latch receptacle and displace the button toward the base and begin insertion of the microneedles into a tissue surface; and (ii) to move the latch into the second latch receptacle and trigger a tactile signal and/or an audible signal that sufficient force has been applied to the microneedle patch to effect full insertion of the microneedles into the tissue surface.

In some embodiments, the button 416, or at least its side surfaces, have a distinctly different color material from the base 414 so that a user may more easily or more quickly identify when the button 416 has fully reached the actuated position.

The microneedle patch 400 optionally may further include a secondary feedback mechanism indicating to the user that enough force has been applied to successfully deliver the microneedles to the tissue, similar to that described with respect to FIG. 1. That is, in some instances, the force required to translate the button my not be sufficient to insert the microneedles into the tissue. The secondary feedback mechanism may be triggered upon application of sufficient to insert the microneedles.

In some embodiments, the microneedle patch 400 further includes a release liner or other material (not shown) covering the recess 412, for example by being releasably adhered to the bottom of backing layer 408, to further protect the microneedles prior to applying the patch. The release line would be removed prior to placing the microneedle patch against the skin.

In some alternative embodiments, the latch and latch receptacle features may be replaced or augmented with other force setting/actuating mechanisms such as plastic snaps, brittle fracture, plastic snap latches deform or snap into recess/pocket.

### Microneedle Storage Systems

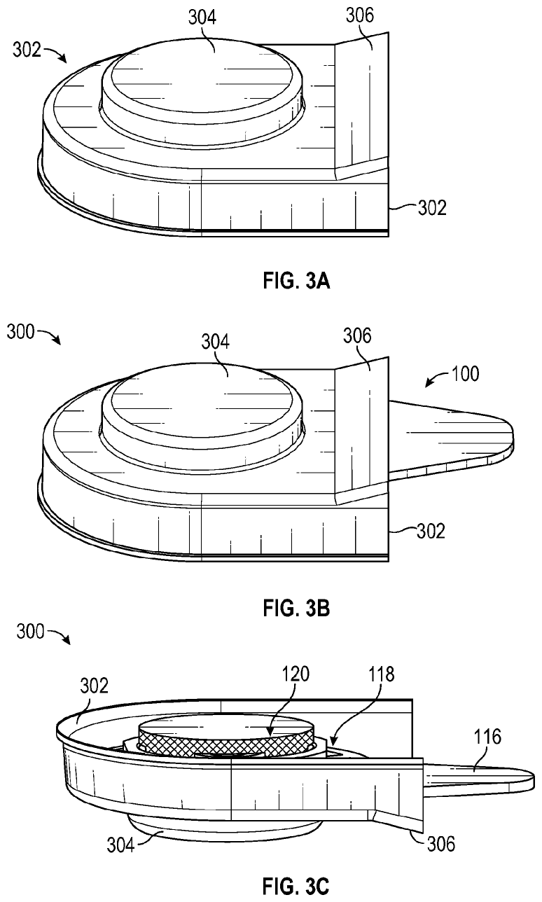

As shown in FIGS. 3A-3C, the microneedle patch 100 may be housed on a tray 300 having an inner surface 302 defining a recessed region 304 therein. The recessed region 304 may be dimensioned to receive and enclose the array of microneedles 104 in a non-contacting manner. The tray 300 may be also be releasably adhered to the microneedle patch 100 to prevent movement of the patch 100 within the tray 300. That is, the adhesive layer (not shown) of the microneedle patch 100 may be releasably secured to the inner surface 302 of the tray 300. Because contact between the tray and microneedle patch is limited substantially to the adhesive layer and/or backing, the integrity of the one or more microneedles is advantageously retained during storage. In addition, the tray may also protect the one or more microneedles from moisture, gases, or other contaminants that could degrade the substance of interest, reduce the shelf life, or diminish the effectiveness of the substance of interest.

In some embodiments, the tab portion 116 of the microneedle patch 100 may extend from the tray 300 to ease removal of the microneedle patch 100 from the tray. The tray 300 may also include a ledge 306 to improve user access to the microneedle patch 100 stored within the tray 300.

The trays may take a variety of shapes and sizes, such as a rectangular shape, a planar shape formed with a cap, or a partial ellipsoidal shape. The tray may further include one or more additional features with various functions or to impart a desired aesthetic to the tray. For example, the tray may include one or more depressions, holes, or cutouts. Such features may facilitate removal of the microneedle patch from the tray. The recessed region for receiving the one or more microneedles also may be positioned in the tray such that at least a portion of the tab extends over the perimeter of the tray.

A variety of materials may be used to make the trays provided herein, non-limiting examples of which include polymers (e.g., polytetrafluoroethylene (PTFE), fluorinated ethylene propylene (FEP), polyvinylidene fluoride (PVDF), polyvinyl fluoride (PVF), polyethylene, or polypropylene), metallized polymers, elastomers, non-woven and woven materials, paper-based materials, foam, metal or foil, and the like. In some embodiments, the tray may be formed of composite materials or multilayer materials. For example, a multilayer material may include one or more layers that impart the desired structural properties and one or more layers that impart the desired moisture and gas barrier properties.

A tray may be configured to house a single patch or a plurality of patches (e.g., 2, 3, 4, 5, 6, 7, 8, 10, 12, or 20 patches, or more or less). The tray may include a plurality of recesses, with each recess corresponding to one of the microneedle patches. The trays may also include one or more lines of weakness (e.g., perforations, score lines, and the like) so that portions of the tray are separable from other portions of the tray. In some embodiments, the patches may be stored on one side of the tray, while in other embodiments, the patches may be stored on only one side of the tray (e.g., within recessed regions on both sides of the tray).

These trays alone may be sufficient to protect patch prior to use; however, additional features may also be used. For example, one or more trays may be disposed in a flexible container (e.g., pouch) and/or rigid container (e.g., box). In some embodiments, a lid may be disposed on the tray to protect the microneedle patch prior to use. Such lids may be the same or a different material from the tray, and may be sealed to the perimeter of the tray (i.e., using a heat seal, cold seal, or pressure sensitive adhesive). In one embodiment, a desiccant may be provided in the recessed regions or in the flexible or rigid container housing the tray. A desiccant may alternatively or in addition be part of the tray itself. For example, a desiccant material may be included (e.g., dispersed in or coated onto) the material forming the structure of the tray. For example, the tray may be formed of a desiccant polymer known in the art.

The trays may formed using a variety of different methods, non-limiting examples of which include various molding methods (e.g., thermoforming, injection molding, stamping, casting), 3-D printing, and the like.

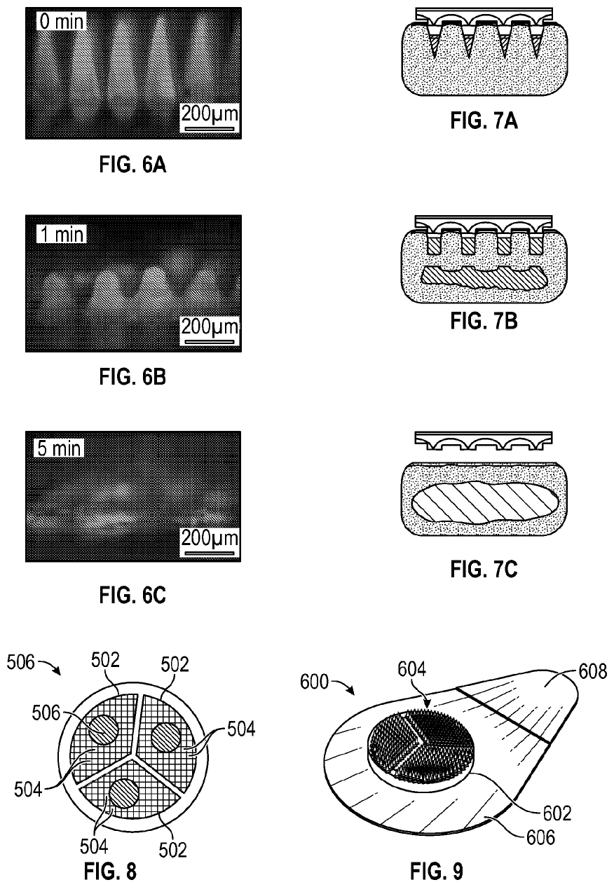

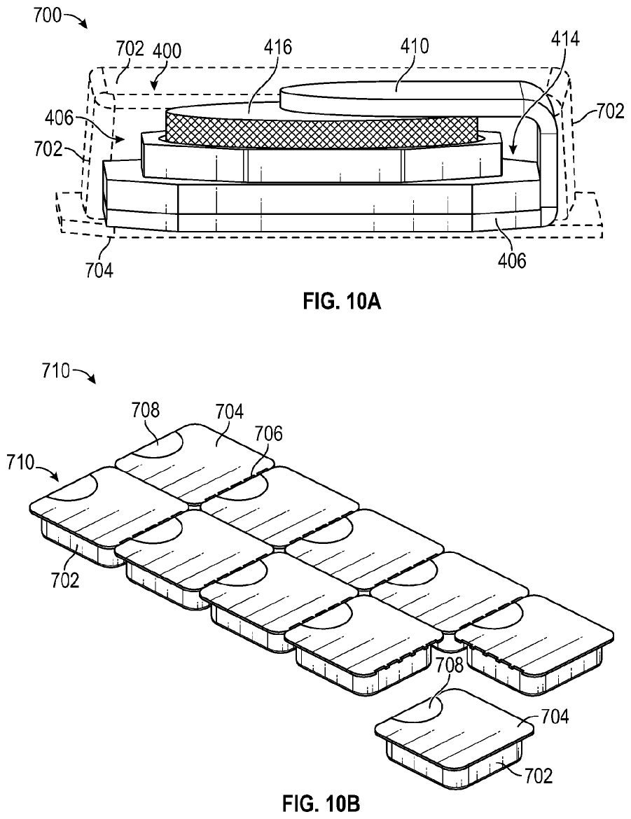

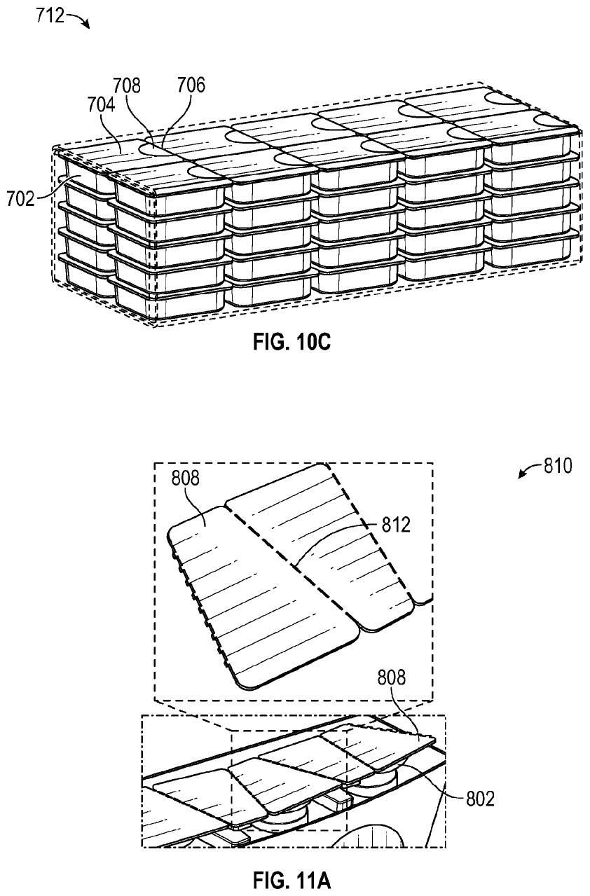

In some embodiments, as shown in FIGS. 10A-10C, the microneedle patches 400 as described with respect to FIGS. 6A-6B may be folded in half and stored in a sealed pouch 700. In some embodiments, as shown in FIG. 10A, the base substrate 408 of the microneedle patch 400 may be folded at the point where the feedback indicator 406 is attached to the base substrate 408, and again proximate to the interface between the tab portion 410 and the base substrate 408. The result is a compact patch 400 that can be more easily contained in a compact container or pouch.

In embodiments, a pouch material (e.g., foil) 702 may be adhered to microneedle patch 400. That is, the microneedle patch 400 may have an adhesive layer (not shown) on the bottom side of the backing layer 408. The pouch material 702 may therefore be adhered to the adhesive surface of the backing layer 408, and folded over on itself to form a pouch 700. In some embodiments, the pouch material 702 may fully surround the microneedle patch 400, i.e., the pouch 700 is fully formed of the pouch material 702. In other embodiments, the pouch material 702 surrounds the microneedle patch 400 on all but one side, which is covered with a lid 704. The lid 704 may be sealed or otherwise attached to the pouch material 702.

As shown in FIG. 10B, a plurality of the trays 700 may be arranged in an array to form a packaging unit 710. The trays 700 within each packaging unit 710 may be releasably attached to each adjacent tray 700 in the packaging unit 710. For example, the edges 706 of each tray 700 may be defined by a line of perforations in a shared sheet material (which forms the lids 704), such that the respective trays 700 may be detached from one another along the edges 706. Each tray 700 may also include an opening tab 708 to facilitate removal of the lid 704. In some embodiments, multiple packaging units 710 may be stacked and stored in a box 712, or other storage container, for long term storage and/or transport of the microneedle patches.

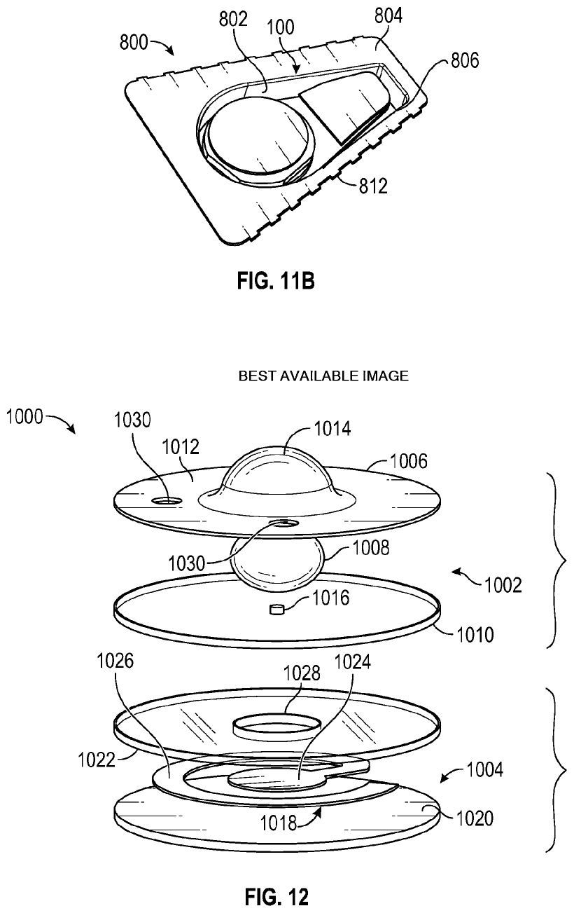

In other embodiments, as shown in FIGS. 11A-11B, the microneedle patch 100 as describe with respect to FIG. 1 may be placed in a tray 800 sized and shaped to receive the microneedle patch 100. The microneedle patch 100 may sit within a cavity 802 defined within a base portion 804 of the tray 800. In some embodiments, the bottom 806 of the cavity 802 is angled so that the tab portion 116 of the microneedle patch 100 may be easily grasped by to remove the patch 100 from the tray 800. That is, the angled bottom 806 of the cavity 802 may angle the tab 116 of the microneedle patch 100 in an upward direction so that it is positioned near the opening of the cavity 802. In some embodiments, the tray 800 may be sealed with a lid 808 that covers at least the cavity 802. However, it is preferred that the lid 808 covers the entire base 804 of the tray 800. The lid 808 may be formed of foil or another suitable film material that may be pealed away from the tray 800 when the patch 100 is ready for use.

As shown in FIG. 11A, a plurality of trays 800 may form a packaging unit 810 where the edge 812 of one tray 800 is releasably attached to the edge 812 of each adjacent tray 800. In some embodiments, the releasably attached edges 812 are defined by a line of perforations in a shared sheet material, i.e., a sheet material used to form the tray 800 lids 808. The packaging units 810 may be stacked and stored in boxes or other storage containers, similar to packaging units 710.

### Microneedle Formulations

A wide range of substances may be formulated for delivery to biological tissues with the present microneedle patches and methods. The microneedles may be formed of one or more substances of interest and one or more excipients. As used herein, the term “substance of interest” includes active pharmaceutical ingredients, allergens, vitamins, cosmetic agents, cosmeceuticals, markers (e.g., colored dyes, inks, pigments, or radiological dyes or markers), and other materials that are desirable to introduce into skin or another a biological tissue. In some embodiments, the substance of interest is a prophylactic, therapeutic, or diagnostic agent useful in medical or veterinary applications. In some embodiment, the substance of interest is a bioactive agent, which may be prophylactic or therapeutic substance, which may be referred to herein as an API. The API may be selected from suitable proteins, peptides and fragments thereof, which can be naturally occurring, synthesized or recombinantly produced. In some embodiments, the substance of interest comprises a vaccine.

The substance of interest may be included in a formulation with one or more excipients and other additives that are used in pharmaceutical formulations. Non-limiting examples of such excipients include stabilizers, buffers, bulking agents or fillers, adjuvants, surfactants, disintegrants, antioxidants, solubilizers, lyo-protectants, antimicrobials, antiadherents, colors, lubricants, viscosity enhancer, glidants, preservatives. The excipients may be those found in existing drug products (such as those listed in the FDA's Inactive Ingredients in Approved Drug Products database) or may be novel, and may be effective to perform more than one function (e.g., a sugar may be used as a stabilizer and a bulking agent, a buffer may be used to both buffer pH and protect the substance of interest from oxidation). The one or more selected excipients desirably improve the stability of the substance of interest during drying and storage of the microneedle patches.

### Methods of Use

The microneedle patches provided herein may be self-administered or administered by another individual (e.g., a parent, guardian, minimally trained healthcare worker, expertly trained healthcare worker, and/or others). Unlike prior art microneedle systems, the microneedle patches provided herein may be directly handled and administered by the person applying the patch without requiring use of an applicator to apply the required force/pressure.

Thus, embodiments provided herein further include a simple and effective method of administering a substance of interest with a microneedle patch. The method may include identifying an application site and, preferably, sanitizing the area prior to application of the microneedle patch (e.g., using an alcohol wipe). If needed, the application site may be allowed to dry before application of the microneedle patch. The patch may be removed from the tray or pouch in which it is releasably secured by grasping the tab portion of the patch between the thumb and finger and peeling the patch from the tray or pouch. The patch then is applied to the patient's skin/tissue and manually pressed into the patient's skin/tissue (e.g., using the thumb or finger) by applying a sufficient pressure to insert the one or more microneedles into the patient's skin/tissue. After administration is complete, the patch may be removed from the patient's skin/tissue by manually grasping the tab portion (e.g., between the thumb and finger), peeling the patch off the patient's skin/tissue, and discarding the patch.

FIGS. 6A-7C depict the application process for the microneedle patches described herein, and the dissolution of the microneedles within the patient's skin/tissue after insertion. For example, as shown in FIGS. 6A and 7A, the microneedle patch may be inserted into the patient's skin/tissue so that a majority of the microneedles are disposed underneath the surface of the skin/tissue. As the microneedles begin to dissolve, as shown in FIGS. 6B and 7B, the tip portion of the microneedles may become fully dissolved and dispersed within the tissue, while the base portion of the microneedle remains intact. However, at the point where the patch is removed from the patient's skin/tissue is removed, as shown in FIGS. 6C and 7C, the microneedles may be fully dissolved within the patient's skin/tissue.

In some embodiments, a user may use one or more indicators prior to, during, and/or after administration of the microneedle patch. Such indicators may be elements incorporated into the microneedle patch that provide a detectable signal or may result from the user performing one or more actions, such as evaluating the microneedle patch or the patient's skin/tissue following administration.

Various indicators may be assessed by a user during application of the patch to signal whether the patch has been properly applied and/or may be removed. For example, in some embodiments an indicator provides a signal that a predetermined threshold force has been reached or that the microneedles have penetrated/punctured the patient's skin, indicating that the user may discontinue applying pressure to the patch. In some other embodiments, the indicator may provide a signal at the end of a hold down period, i.e., a period of time after insertion throughout which the patient or user must continue to apply pressure to the microneedle patch. The hold down period may have a duration between 0 seconds to 120 seconds, such as between 0 seconds to 60 seconds, 0 seconds to 30 seconds, or 0 seconds to 10 seconds.

The above-described indicators and feedback also may function to provide evidence that the microneedle patch has already been used, and may be helpful in situations in which the patch is not properly discarded after use (i.e., thereby avoiding attempts to reuse the patch, which would result in an ineffective treatment, or potential exposure to a bio-hazardous material that has been contaminated by the previous patient's bodily fluids). Evidence of use of microneedle patches is particularly helpful because the microneedles are small structures that are barely visible with the naked eye.

### Manufacture

Methods for manufacturing microneedle patches and systems also are provided. Such methods preferably are performed under a minimum ISO 7 (class 10,000) process or an ISO 5 (class 100) process. In some embodiments, the manufacture of solid, dissolvable microneedles includes filling a negative mold of the microneedles with an aqueous or non-aqueous casting solution of the substance of interest and then drying the casting solution to provide the solid microneedles. The filling and dry step may be repeated with the same or a different casting solution. In some embodiments, droplets of the casting solution may be deposited onto the mold, or a portion thereof. The droplets may then be dispersed throughout the mold.

In some embodiments, the mold contains a single opening onto which a droplet can be deposited, and said droplet will disperse across all of the microneedle cavities extending from said opening. In other embodiments, as shown in FIG. 8, the mold 500 may have several openings 502, each opening defining a plurality of microneedle cavities 504 therein. A droplet 506 of a casting solution may be deposited onto each segment 502 of the mold 500. The droplets 506 may be of the same or different casting solutions. That is, in some instances, each of the droplets 506 are of the same casting solution such that the resulting microneedle patch has an array of microneedles all having the same formulation. However, in other instances, the droplets 506 can be of different casting solutions such that the resulting microneedle array contains microneedles having two or more different formulations.

An exemplary microneedle patch 600 formed from a segmented mold, such as the mold of FIG. 8, is depicted in FIG. 9. The microneedle patch 600 may include a base substrate 602 and a segmented array of microneedles 604 extending therefrom. The patch 600 may also include a backing layer 606 to which the base substrate 602 is attached, and a tab portion 608 extending from the backing layer 606, similar to microneedle patch 100 described with respect to FIG. 1.

While FIGS. 8-9 depict a mold and microneedle patch having three segments, it would be understood that any number of segments may be possible. For example, the mold could have 2, 4, 5, 6, 8, or 10 segments, or any other desirable number thereof.

In some embodiments, it may be desirable to use a multi-step casting process to form the microneedles and base substrate. For example, the tips of the microneedles may be partially filled in a first step with a casting solution comprising the substance of interest (and one or more excipient (matrix) materials) followed by one or more subsequent fill steps with casting solutions of bulking materials (e.g., carboxymethylcellulose sodium, polyvinyl alcohol, sugars, gelatin, polyvinylpyrrolidone (PVP), celluloses, and/or other matrix materials including non-dissolving materials such as urethanes or acrylics) with or without the same or a different substance of interest. After filling and at least partially drying the microneedles in the negative mold, the adhesive layer and backing layer may be applied to the base substrate prior to removing the microneedles from the mold. In some embodiments, the adhesive layer and/or backing layer are pre-formed prior to application to the base substrate, while in other embodiments the adhesive layer and/or backing layer may be formed directly in-line. After at least partially drying the microneedles, the microneedles may be removed from the mold. For example, the microneedles may be removed from the mold before fully dry (e.g., when still in a rubbery state), but when strong enough to be peeled, and then dried further once removed from the mold to further solidify/harden the microneedles. In such embodiments, the microneedles may complete drying prior to or after packaging.

The microneedle patches may then be attached to the trays and undergo one or more additional packaging steps. For example, the microneedle patches may be applied to the tray and packed in a foil pouch with desiccant under aseptic conditions.

### Microneedle Wear Time Indicators

Feedback indicators may also provide information to the user (and/or patient) that the microneedle patch has been worn for a sufficient amount of time (i.e., that the substance of interest has been released into the target tissue). Such indicators may be especially useful to provide a user confidence that the substance of interest was effectively delivered, particularly where delivery of the substance of interest is dependent upon insertion and dissolution of the microneedles or coating. The indicator may measure full or partial microneedle dissolution, depending on whether full or partial microneedle dissolution is needed for delivery of an effective amount of the substance of interest. For example, by measuring full dissolution, the indicator can signal to the user that the microneedle patch can be removed from the patient's skin. It may also be useful in some circumstances for the indicator to signal partial dissolution if the partial dissolution would be sufficient to provide an effective amount of the substance of interest or to otherwise signal that user interaction with the microneedle patch is necessary or desirable.

An exemplary wear time indicator 1000 is shown in FIG. 12. The wear time indicator (WTI) 1000 may provide the user with a visible indication that the microneedle patch has been worn on the patient's skin for a sufficient period of time. In embodiments, the WTI 1000 includes a dye blister 1002 and a wick assembly 1004. The dye blister 1002 may include a depressible casing 1006 containing a burstable dye reservoir 1008. The depressible casing 1006 may include a substantially flat portion 1012 and a deformable portion 1014 under which the dye reservoir 1008 is placed. In use, the patient or user may depress the deformable portion 1014 of the depressible casing 1006 with enough force to burst the dye reservoir 1008 therein. After the dye reservoir 1008 has been burst, the dye within from the dye reservoir 1008 may be transferred to the wick assembly 1004 through a passage 1016 in the bottom 1010 of the depressible casing 1006 at a controlled rate. That is, the size of the passage 1016 may be selected so that the dye diffuses at a predetermined, controlled rate.

The wick assembly 1004 may include a wicking membrane 1018 configured to absorbed dye from the dye blister 1002. The wicking membrane may be mounted on a backing 1020 and covered by a protective layer 1022. In some embodiments, the backing 1020 itself may be formed of an adhesive material so that the wicking membrane 1018 can be secured directly to the adhesive surface of the backing 1020. In other embodiments, the wicking membrane 1018 is attached to the backing 1020 with an additional adhesive (not shown), or the protective layer 1022 is effective to retain the wicking membrane 1018 in an appropriate position on the backing 1020. The backing layer 1020 may also contain an additional adhesive layer (not shown) on the side opposite the wicking membrane 1018 to secure the wear time indicator to a microneedle patch, such as those described herein.

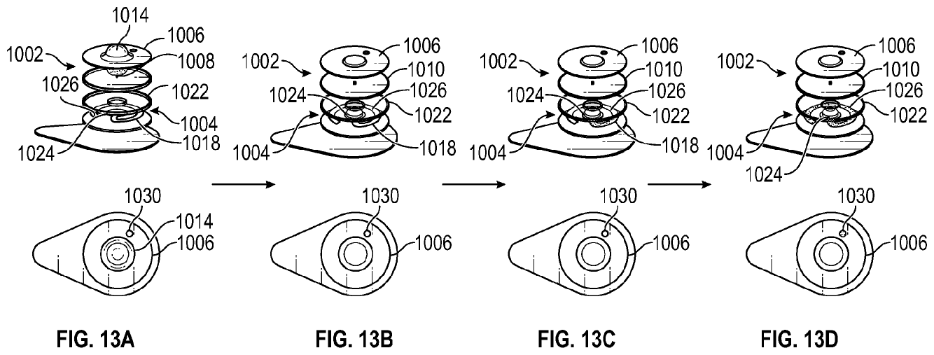

In embodiments, the wicking membrane 1018 has a center portion 1024 onto which dye from the dye blister 1002 is initially deposited, and a peripheral portion 1026 along which the dye will travel over a given period of time. In some embodiments, as shown in FIG. 12, the center portion 1024 is circular and positioned beneath a similarly sized and shaped opening 1028 in the protective layer 1022 of the wick assembly 1004. As dye from the dye reservoir 1012 passes through the opening 1028 in the protective layer 1022, onto the center portion 1024 of the wicking membrane 1018. As the center portion 1024 becomes saturated with dye, the dye will begin to travel along the peripheral portion 1026 of the wicking membrane 1018 disposed around the center portion 1024 in a spiral-like configuration. As time passes, the dye will travel around the peripheral portion 1026 of the wicking membrane 1018, where the distance the dye has traveled, or the portion of the wicking membrane 1018 onto which the dye has traveled, (i.e., the amount of dye that has been absorbed) corresponds with an amount of time that the microneedle patch has been worn. For example, as shown in FIGS. 13A-13D, the wicking membrane 1018 may be free of dye before the dye reservoir 1012 is broken (FIG. 13A), and after the reservoir 1012 is broken the dye will cover the center portion 1024 (FIG. 13B) and part of the peripheral portion 1026 (FIG. 13C). At the end of the designated wear time, the dye will have covered the entire peripheral portion (FIG. 13D).

The upper portion 1008 of the dye blister 1002 may also include one or more windows 1030 through which one or more regions of the wicking membrane 1018 are visible. The visible region of the wicking membrane 1018, or the position of the window 1030, may depend on the desired wear time of the patch. For example, if the optimal wear time for a patch is 10 minutes, the dye may take 10 minutes to be fully absorbed by the wicking membrane 1018. In some embodiments, the dye blister 1002 has a single window 1030 for indicating the final wear time for the microneedle patch, as shown in FIGS. 13A-13D. In other embodiments, as shown in FIG. 12, the dye blister 1002 may have at least one additional window 1030 positioned at an intermediate location along the peripheral portion 1026 of the wicking membrane 1018 to indicate a shorter wear time than the total wear time. For example, if the total wear time is 10 minutes, a first window 1030 may be placed at the end of the peripheral portion 1026 of the wicking membrane 1018 to indicate the full 10 minute wear time, and a second window 130 may be placed to indicate a shorter wear time, e.g., 1 minute, 3 minutes, 5 minutes, etc. In embodiments, the wear time is from 30 seconds to 10 minutes, preferably 30 seconds, 1 minute, 3 minutes, or 5 minutes.

Modifications and variations of the methods and devices describe herein will be obvious to those skilled in the art from the foregoing detailed description. Such modifications and variations are intended to come within the scope of the appended claims.

1. A microneedle patch comprising:
an array of microneedles;
a base substrate having a first side from which the microneedles extend and an opposing back side; and
a force feedback indicator (FFI) attached to the back side of the base substrate, wherein the FFI comprises a base and a button, which has an upper surface and a side surface, wherein the button is configured to translate from a pre-actuated position into an actuated position within the base, wherein the side surface is substantially visible in the pre-actuated position and substantially not visible in the actuated position.

2. The microneedle patch of claim 1, wherein the FFI is configured to further provide tactile and/or audible confirmation of sufficient force applied to microneedle patch to effect insertion of the microneedles.

3. The microneedle patch of claim wherein the side surface has different color than the color of the upper surface, and/or wherein the side surface has different color than the color of the base.

4. (canceled)

5. The microneedle patch of claim 1, wherein the base has a lateral ring portion and a central cup portion, wherein a snap dome is disposed between a concavity of the cup portion and the button.

6. The microneedle patch of claim 1, wherein the button further comprises at least one latch leg extending from the side surface in a direction away from the upper surface, the latch leg comprising a lip configured to lock with the base in the actuated position to prevent return of the button to the pre-actuated position.

7-8. (canceled)

9. The microneedle patch of claim 1, further comprising a tape layer comprising an adhesive face and a handling tab, wherein the tape layer is secured to the FFI.

10. The microneedle patch of claim 9, wherein the tape layer comprises an aperture through which the central cup portion of the base extends, and the FFI is attached to the back side of the base substrate on a raised surface of the central cup portion opposite of the concavity and/or wherein the tape layer is secured to the lateral ring portion on a side opposite to the adhesive face, and wherein the adhesive face surrounds the aperture and the microneedle array and is configured to be removable adhered to a patient's skin.

11. (canceled)

12. The microneedle patch of claim 1, wherein the FFI is configured to lock the button into the actuated position when a force applied to the patch by a user to insert the microneedles meets or exceeds a predetermined threshold.

13. The microneedle patch of claim 12, wherein the button comprises a latch configured to lock into the base in the actuated position to prevent return of the button to the pre-actuated position following application of a predetermined microneedle insertion force to the button.

14. The microneedle patch claim 1, wherein the FFI is configured to lock the button into the actuated position when a force applied to the patch by a user to insert the microneedles is effective to cause the button to be displaced into the microneedle patch a predetermined distance.

15-22. (canceled)

23. The microneedle patch of claim 1, further comprising a handling tab attached directly to a lower surface of the base.

24. The microneedle patch of claim 23, wherein the handling tab has a storage position in which the handling tab is folded over a top surface of the button and can be unfolded for use of the microneedle patch.

25. The microneedle patch of claim 24, further comprising a release liner covering the microneedle array and being releasably adhesively attached to the handling tab so that the release liner is also folded over the button and can be removed for use of the microneedle patch.

26. The microneedle patch of claim 1, wherein at least an upper portion of the button in the pre-actuated position is elevated above the base and in the actuated position is flush with or recessed into the base.

27. The microneedle patch of claim 1, wherein the base substrate and array of microneedles are attached only to the button of the FFI, and wherein the base of the FFI is dimensioned such that in the pre-actuated position, the array of microneedles is in a position recessed in an opening in a lower surface of the base of the FFI.

28. The microneedle patch of claim 26, wherein:
the button of the FFI comprises a latch;
the housing comprises a first latch receptacle, which is configured to receive the latch and releasably hold the button in the pre-actuated position; and
the housing comprises a second latch receptacle, which is configured to receive the latch and unreleasably hold the button in the actuated position.

29-30. (canceled)

31. A microneedle patch packaging system, comprising:
at least one of the microneedle patches of claim 1, which has an adhesive surface; and
a foil or other pouch material adhered to the adhesive surface,
wherein the foil or other pouch material is folded over and sealed to form a sealed pouch enclosing the at least one of the microneedle patches.

32. A microneedle patch packaging unit, comprising:
the microneedle patch of claim 1, which has a handling tab;
a packaging tray having a cavity in which the microneedle patch is disposed; and
a foil or other film attached to the tray to seal the cavity,
wherein the packaging system is configured such that upon removal of the foil or other film from the tray, the handling tab is positioned toward the opening of the cavity to facilitate ease of grasping the handling tab to remove the microneedle patch from the packaging tray.

33-34. (canceled)

35. A microneedle patch comprising:
an array of dissolvable microneedles;
a base substrate having a first side from which the microneedles extend and an opposing back side; and
a wear time indicator (WTI) which is attached to the back side of the base substrate and which is configured to provide a visual indication that the microneedle patch has been worn on a user's skin for a period sufficient to effect dissolution of the microneedles following their insertion into the user's skin, the period being a wear time.

36. The microneedle patch of claim 35, wherein the WTI comprises a dye blister and a wick assembly, wherein the dye blister comprises a burstable dye reservoir configured to release a dye into the wick assembly upon application of a force to the microneedle patch to effect insertion of the microneedles, a position of the dye within the wick assembly corresponding to the wear time.

37-45. (canceled)

46. A microneedle patch comprising:
an array of microneedles;
a base substrate having a first side from which the microneedles extend and an opposing back side; and
a force feedback indicator (FFI) comprises a base and a button, wherein the base substrate and array of microneedles are attached only to the button of the FFI,
wherein the button is configured to translate from a pre-actuated position into an actuated position within the base and wherein the base of the FFI is dimensioned such that in the pre-actuated position, the array of microneedles is in a position recessed in an opening in a lower surface of the base of the FFI.

47-54. (canceled)

55. A microneedle patch packaging system, comprising:
at least one of the microneedle patches of claim 46, which has an adhesive surface; and
a foil or other pouch material adhered to the adhesive surface,
wherein the foil or other pouch material is folded over and sealed to form a sealed pouch enclosing the at least one of the microneedle patches.

56. A microneedle patch packaging unit, comprising:
the microneedle patch of claim 46, which has a handling tab;
a packaging tray having a cavity in which the microneedle patch is disposed; and
a foil or other film attached to the tray to seal the cavity,
wherein the packaging system is configured such that upon removal of the foil or other film from the tray, the handling tab is positioned toward the opening of the cavity to facilitate ease of grasping the handling tab to remove the microneedle patch from the packaging tray.

57. A packaging system comprising a plurality of the microneedle patch packaging units of claim 56, wherein an edge of the packaging tray of each packaging unit is releasably attached at an edge of at least one other packaging tray of another packaging unit.

58-60. (canceled)

61. A microneedle patch packaging system, comprising:
a microneedle patch, which has an adhesive surface; and
a foil or other pouch material adhered to the adhesive surface,
wherein the foil or other pouch material is folded over and sealed to form a sealed pouch enclosing the at least one of the microneedle patches.

62. A microneedle patch packaging unit, comprising:
a microneedle patch, which has a handling tab;
a packaging tray having a cavity in which the microneedle patch is disposed; and
a foil or other film attached to the tray to seal the cavity,
wherein the packaging system is configured such that upon removal of the foil or other film from the tray, the handling tab is positioned toward the opening of the cavity to facilitate ease of grasping the handling tab to remove the microneedle patch from the packaging tray.

63. A packaging system comprising a plurality of the microneedle patch packaging units of claim 62, wherein an edge of the packaging tray of each packaging unit is releasably attached at an edge of at least one other packaging tray of another packaging unit.

64-66. (canceled)
